(19) United States
(12) Patent Application Publication
(10) Pub. No.: US 2024/0206722 A1
(43) Pub. Date: Jun. 27, 2024

(54) Methods and Systems for Optimizing Refractive Refraction of Human Eyes

(71) Applicants: Junzhong Liang, Fremont, CA (US); Ling Yu, Fremont, CA (US)

(72) Inventors: Junzhong Liang, Fremont, CA (US); Ling Yu, Fremont, CA (US)

(21) Appl. No.: 18/557,796

(22) PCT Filed: Apr. 27, 2022
(86) PCT No.: PCT/US2022/026459

§ 371 Date: Oct. 27, 2023

Related U.S. Application Data
(60) Provisional application No. 63/258,486, filed on May 6, 2021.

(51) Int. Cl. A61B 3/028; A61B 3/10; A61B 3/103

(52) U.S. Cl. CPC A61B 3/028; A61B 3/1015; A61B 3/103

(57) ABSTRACT

Methods and systems are disclosed for optimizing refractive prescriptions of human eyes. First, objective refraction devices such as aberrometers and auto-refractors will not only provide an objective estimate of sphero-cylinder correction, but also a quality metrics for at least one of a) measuring the confidence level in the objectively determined cylinder power and cylinder axis in addition to the objective sphero-cylinder correction, b) assessing/displaying quality of vision corrections for a plurality of cylinder power. Second, the quality metrics will be used to elect one of a plurality of modes of subjective refraction with a phoropter: 1) one mode for the subjective determination of spherical power only, 2) another mode for subjective determination of both sphere power and cylinder power.

### RELATED APPLICATIONS

This application to U.S. Provisional Patent Application No. 63/258,486, filed on May 3, 2021 by Junzhong Liang and Ling YU, entitled “METHODS AND SYSTEMS FOR OPTIMIZING REFRACTIVE REFRACTION OF HUMAN EYES,” all of which are hereby incorporated by reference in full.

### BACKGROUND

Conventional refraction processes rely on the experience and skills of an individual eye care professional (e.g., an optometrist or optician) to set the starting and ending points of a spherical power, a cylinder power, and a cylinder axis for an eyeglass prescription.

A block diagram 10 representing a conventional refraction process is shown in FIG. 1. First, an autorefractor 11 is typically used to take an objective measurement of an eye's refractive errors and provide a rough objective prescription in objective refraction step 12, where the objective prescription includes an objective spherical power Fₛ, an objective cylinder power F_{c} and an objective cylinder angle Fₐ. Second, an eye care professional determines a rough spherical correction in a phoropter 13, and then administrates a subjective optimization of spherical power, cylinder power and cylinder angle based on the objective prescription from step 12. The subjective optimization is based on the experience and skill of the optometrist or optician, and on subjective feedback of the tested subject (i.e., the patient). The autorefractor 11 can also be a wavefront aberrometer that measures all aberrations in the eye including the objective prescription as well as other aberrations that are not correctable by spherical lenses or torical lenses, including coma, spherical aberration, and other Zernike aberrations.

Steps 16, 17 and 18 are part of the subjective refraction performed using the phoropter 13. In step 16, the cylinder angle Fₐ is subjectively optimized by letting the tested subject first see an astigmatism chart and then an acuity chart afterwards. The eye care professional will set and modify the cylinder angle by an amount □Fₐ based on the objective prescription of step 12 as well as feedback of the tested subject. In step 17, the cylinder power F_{c} is subjectively optimized by having the tested subject view an acuity chart, and an eye care professional will set and modify the cylinder power by an amount □F_{c} based on the objective prescription as well as feedback of the tested subject. In step 18, the spherical power is subjectively optimized by letting the tested subject see an acuity chart, and an eye care professional will set and modify the spherical power Fₛ by an amount □Fₛ based on feedback of the tested subject. The same process of steps 16, 17 and 18 are repeated for the other eye of the tested subject. In subjective refraction step 14, a final prescription of the eyeglasses is determined for each eye using the subjectively optimized spherical power Fₛ+□Fₛ of step 18, the subjectively optimized cylinder power F_{c}+□F_{c} of step 17, and the subjectively optimized cylinder angle Fₐ+□Fₐ of step 16.

This subjective approach is the golden standard for obtaining refractive prescription for human eyes despite many well-known disadvantages. First, it is time-consuming and often takes 15 minutes to 30 minutes for the refraction of two eyes. Second, refraction outcomes depend on skill of individual optometrists, ophthalmologists, or professionals that administrates the refraction process in certain countries. Third, resolution of the lens prescriptions depends on the phoropters used in the process. Cylinder power less than 0.5 D for an eye is usually not corrected while the incremental steps for the spherical power and the cylinder power is 0.25 D.

Consequently, although many configurations and methods for vision correction are known in the art, these conventional methods and systems suffer from one or more disadvantages.

### SUMMARY

In some embodiments, a method for determining refractive corrections of human eyes, the method comprising the steps of: obtaining an objective refraction of an eye of a patient using an objective refraction device, wherein the objective refraction does not involve any subjective feedback from tested subjects and it includes at least an objective sphero-cylinder prescription consisting of an objective spherical power (SPH_o), an objective cylinder power (CYL_o), and an objective cylinder axis (AXIS_o); determining a quality metrics for at least one of 1) measuring the confidence level in the objectively determined cylinder power and cylinder axis in addition to the objective sphero-cylinder correction, 2) assessing/displaying quality of vision corrections for a plurality of cylinder power; using the quality metrics to perform a subjective refraction with a phoropter in a plurality of modes: I) one mode for the subjective determination of spherical power only, II) one mode for the subjective determination of both sphere power and cylinder power.

In some embodiments, a system for determining refractive corrections of human eyes, comprising: an objective aberrometer module configured to obtain an objective measurement of a total wave aberration of an eye of a patient, wherein the objective measurement does not involve responses from the patient; an software module for determining from the total wave aberration of an eye, I) an objective sphero-cylindrical correction that includes an objective spherical power (SPH_o), an objective cylinder power (CYL_o), an objective cylinder axis (AXIS_o), II) a quality metrics for at least one of a) measuring the confidence level in the objectively determined cylinder power and cylinder axis in addition to the objective sphero-cylinder correction, b) assessing/displaying quality of vision corrections for a plurality of cylinder power.

In some embodiments, an improved auto-refactor system for determining refractive correction of human eyes, comprising: a measurement module configured to obtain an objective measurement of an objective sphero-cylindrical correction that includes an objective spherical power (SPH_o), an objective cylinder power (CYL_o), an objective cylinder axis (AXIS_o); an optimization module for performing and generating a profile of quality of vision as a function of a plurality of cylinder powers near the objective cylinder power (CYL_o).

### BRIEF DESCRIPTION OF THE DRAWINGS

FIG. 1 shows a block diagram of a conventional refraction process.

FIG. 2 shows a flow chart of a method for obtaining refractive correction of human eyes in the prior art.

FIG. 3 shows a flow chart for an improved method for obtaining refractive correction of eyes according to the present invention.

FIG. 4 shows calculated Strehl ratio of normalized point-spread function of 4 individual eyes as a function of cylinder powers near the objective cylinder power (CYL_o), and Strehl ratio was calculated from the residual aberrations.

FIG. 5 shows calculated Strehl ratio of normalized point-spread function of 4 other individual eyes as a function of cylinder powers near the objective cylinder power (CYL_o), and Strehl ratio was also calculated from the residual aberrations.

FIG. 6 shows a system for determining refractive correction of human eyes that include a wavefront device and a phoropter according to the present invention.

FIG. 7 shows a system for determining refractive correction of human eyes that include an improved auto-refractor and a phoropter according to the present invention.

### DETAILED DESCRIPTION

Reference now will be made in detail to embodiments of the disclosed invention, one or more examples of which are illustrated in the accompanying drawings. Each example is provided by way of explanation of the present technology, not as a limitation of the present technology. In fact, it will be apparent to those skilled in the art that modifications and variations can be made in the present technology without departing from the spirit and scope thereof. For instance, features illustrated or described as part of one embodiment may be used with another embodiment to yield a still further embodiment. Thus, it is intended that the present subject matter covers such modifications and variations as come within the scope of the appended claims and their equivalents.

Refraction corrections for eyeglasses are typically represented by a spherical power and an astigmatism. In this disclosure, spherical power (“SPH” in the present embodiments) may also be referred to as a focus error or focus power. The astigmatism (AST) includes a cylinder power (“CYL” in the present embodiments) and a cylinder axis (“AXIS” in the present embodiments), where the cylinder axis may also be referred to as a cylinder angle.

Wavefront aberrometers are known to provide objective and precise measurements of all the aberrations in human eyes. An eye's aberrations cause retinal image blur and degrade image quality and visual acuity. Refractive correction for eyeglasses involves the determination of the aberrations in the eye that can be incorporated into corrective eyeglasses. For prescriptions of eyeglasses, an objective sphero-cylinder correction is usually determined, including an objective spherical power SPH_o, an objective cylinder power CYL_o, and an objective cylinder axis AXIS_o.

The objective sphero-cylinder corrections are normally determined by minimizing the residual RMS (Root Mean Square) wavefront error from the objective measurement of a wave aberration of an eye of a patient (W(x,y)).

More advanced algorithms for determine the objective sphero-cylinder correction has also be proposed: 1) numerically varying all three parameters of SPH_o, CYL_o, and AXIS_o in a plurality of combinations, 2) calculating an objective retinal image quality for each combination in the plurality of the combinations, and 3) determining a combination of SPH_o, CYL_o, and AXIS_o to achieve the best image quality (i.e., the best objective retinal image quality). The optimization is performed in an automated manner, where the many combinations of SPH_o, CYL_o, and AXIS_o can be computed quickly by a computer processor. It was also proposed that objective retinal image quality is measured by one or more of the following parameters: a Strehl ratio (peak intensity) of a point-spread function, a half-height width of a point-spread function, or a modulation transfer function at a spatial frequency.

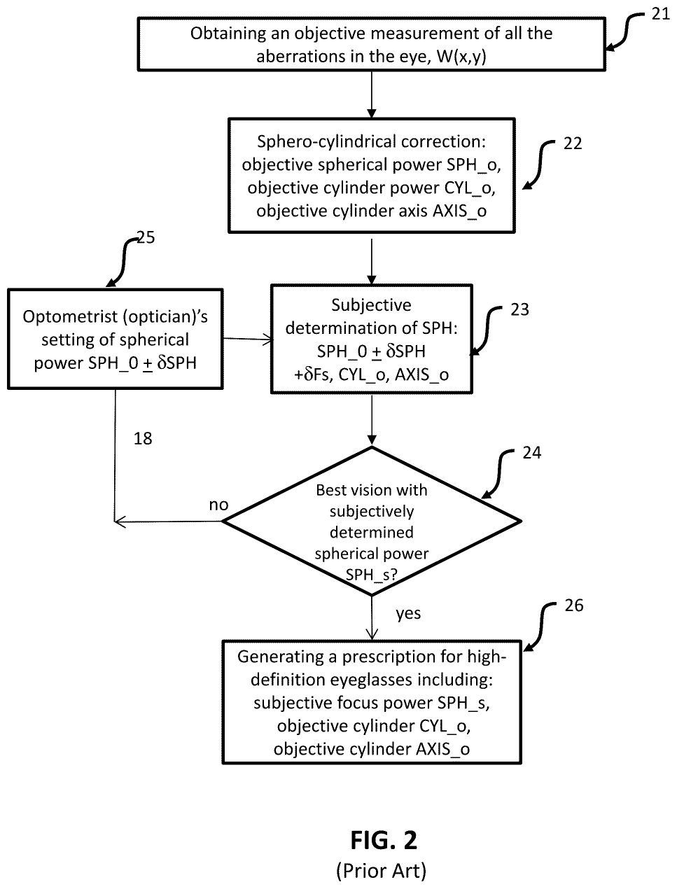

Taking advantages of wavefront aberrometers in precision and speed, a new and unconventional approach of subjective refraction has been proposed and is shown in FIG. 2. This new approach of subjective refraction does not allow optimization of cylinder power and cylinder axis subjectively with inputs from the tested subjects.

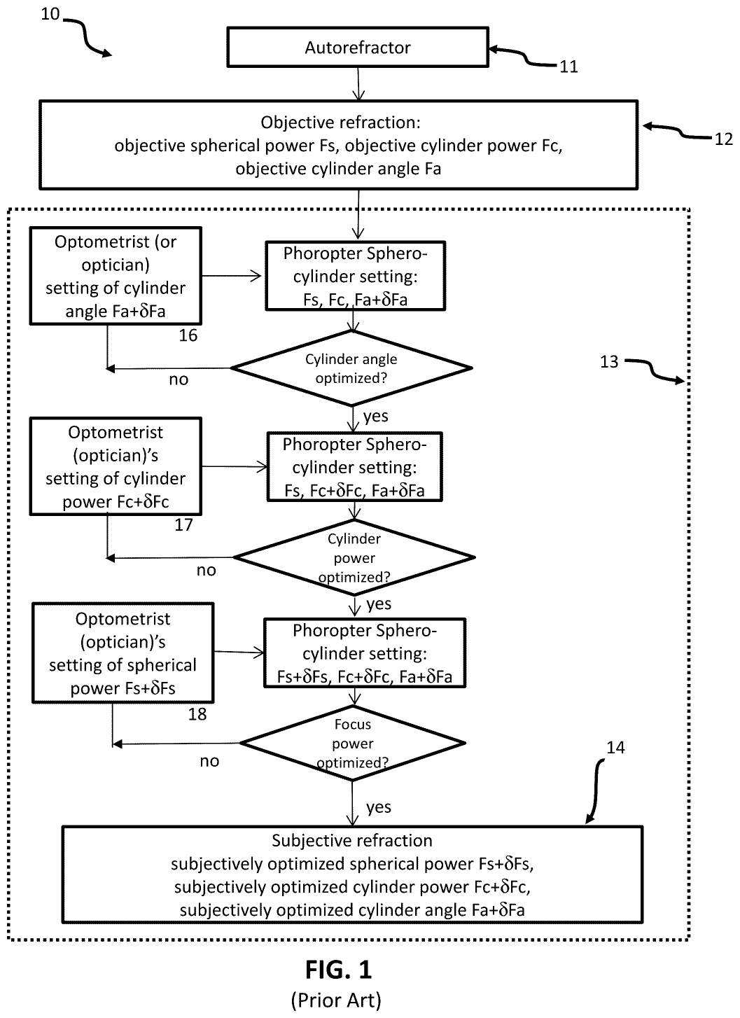

The new method in FIG. 2 was evaluated clinically in comparison to the convention methods in FIG. 1. The approach in FIG. 2 showed many advantages over the conventional refraction in FIG. 1. First, the new approach was significantly less time-consuming and less dependent of the skills of professionals in administrating the test, because 2 out of three variables are objectively determined and are no longer optimized subjectively. Second, the new approach allowed more precise correction of eye's astigmatism because cylinder power with incremental steps of finer than 0.25 D can be prescribed and cylinder power less than 0.5 D (e.g. ⅜ D) can be precisely measured by wavefront aberrometer and corrected as well. Even for standard eyeglasses with incremental step of 0.25 for SPH and CYL, the new approach in FIG. 2 was found better than the conventional refraction in FIG. 1 for majority of patient.

However, it was also found that the approach in FIG. 2 is not perfect, and has issues in accuracy and reliability. For some eyes, the conventional manifest refraction was still found to provide better vision than the new approach in FIG. 2.

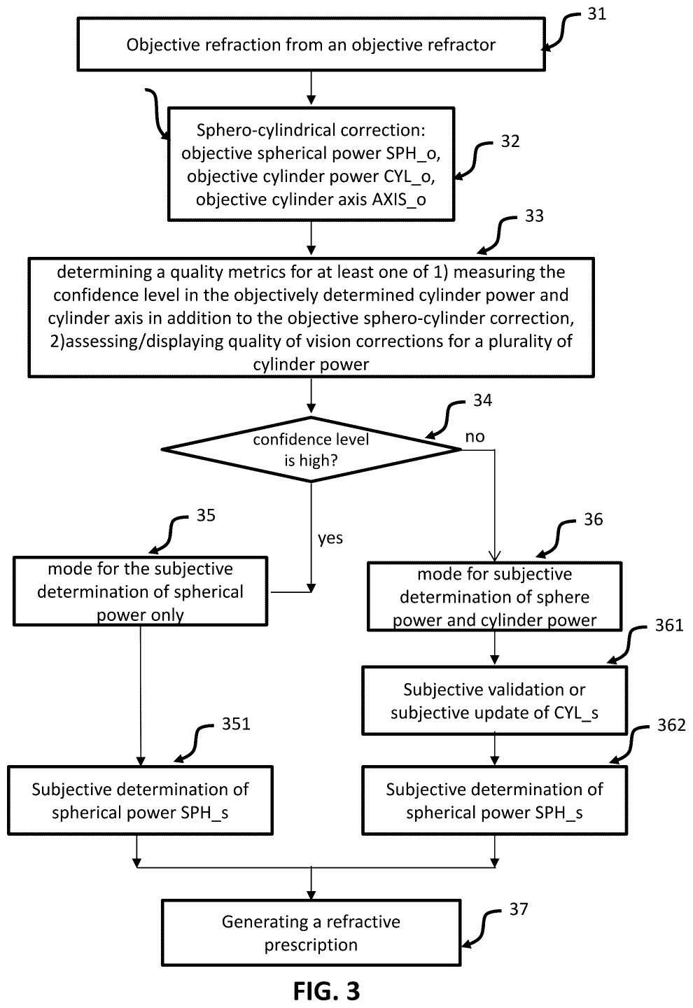

In order to address this problem, we propose an improved method for obtaining refractive correction of eyes, shown in FIG. 3, according to the present invention. The method comprises the steps of: 1) obtaining an objective refraction of an eye of a patient using an objective refraction device 31, and the objective refraction does not involve any subjective feedback from tested subjects and it includes at least an objective sphero-cylinder prescription 32 consisting of an objective spherical power (SPH_o), an objective cylinder power (CYL_o), and an objective cylinder axis (AXIS_o), 2) determining a quality metrics for at least one of 2a) measuring the confidence level in the objectively determined cylinder power and cylinder axis in addition to the objective sphero-cylinder correction, 2b) assessing/displaying quality of vision corrections for a plurality of cylinder power 33, 3) using the quality metrics to guide a subjective refraction with a phoropter in a plurality of modes: one mode for the subjective determination of spherical power only 35 and 351, another mode for the subjective determination of both sphere power and cylinder power 36, 361,362.

In one embodiment, the objective refraction device is a wavefront aberrometer that provides an objective measurement of a total wave aberration of an eye of a patient. The total wave aberration includes the objective sphero-cylindrical correction as well as eye's residual aberrations that are not corrected by the objective sphero-cylindrical correction.

In another embodiment, the quality metrics for measuring the confidence level is measured by a profile of Strehl ratio of eye's point-spread function as a function of a plurality of cylinder powers near the objective cylinder power (CYL_o), and the Strehl ratio is the peak intensity of normalized point-spread function and calculated from the residual aberration. Thus profile of Strehl ratio can be further displayed as well as used for an operator to determine the confidence level in the objectively determined cylinder power and cylinder axis. The confidence level is determined either automatically with an algorithm or by an operator subjectively.

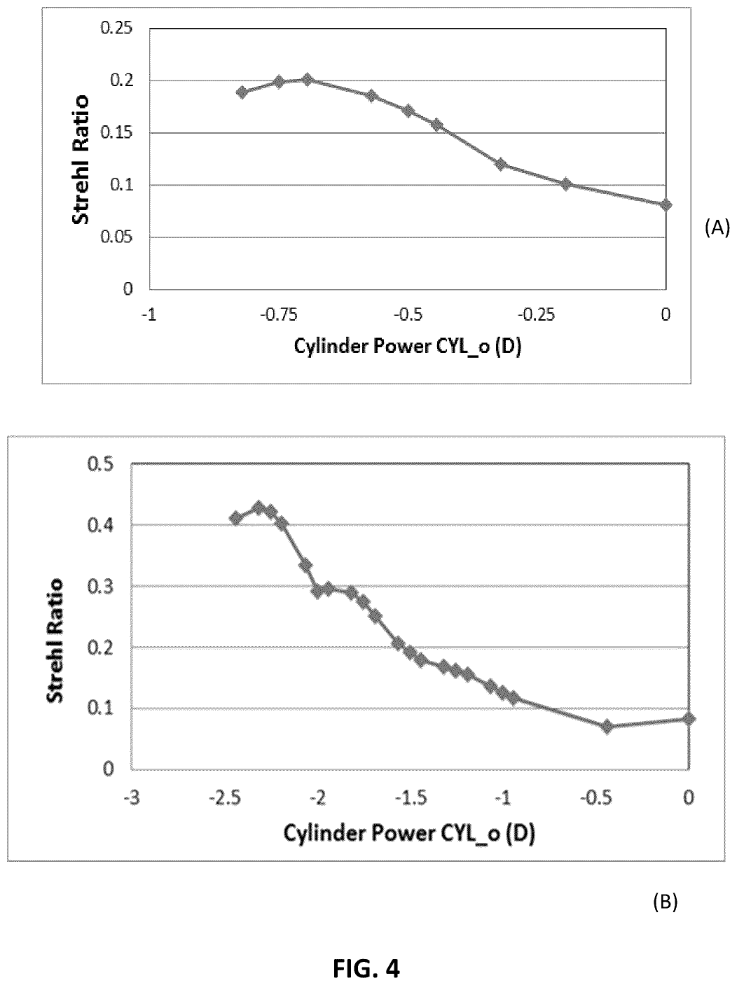

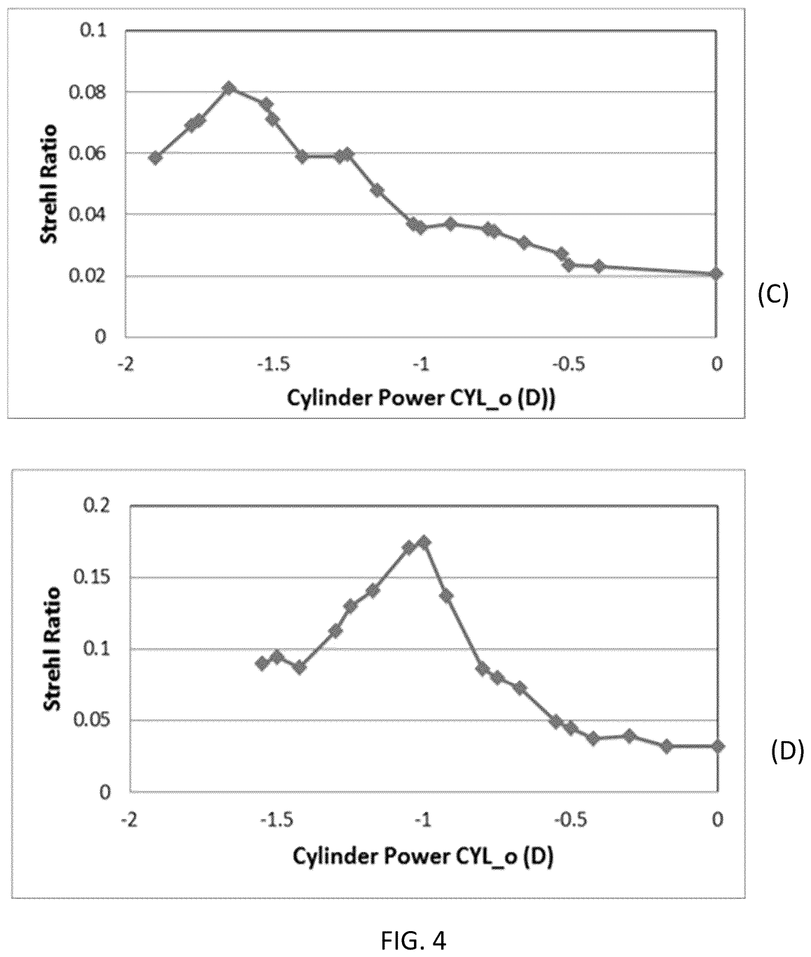

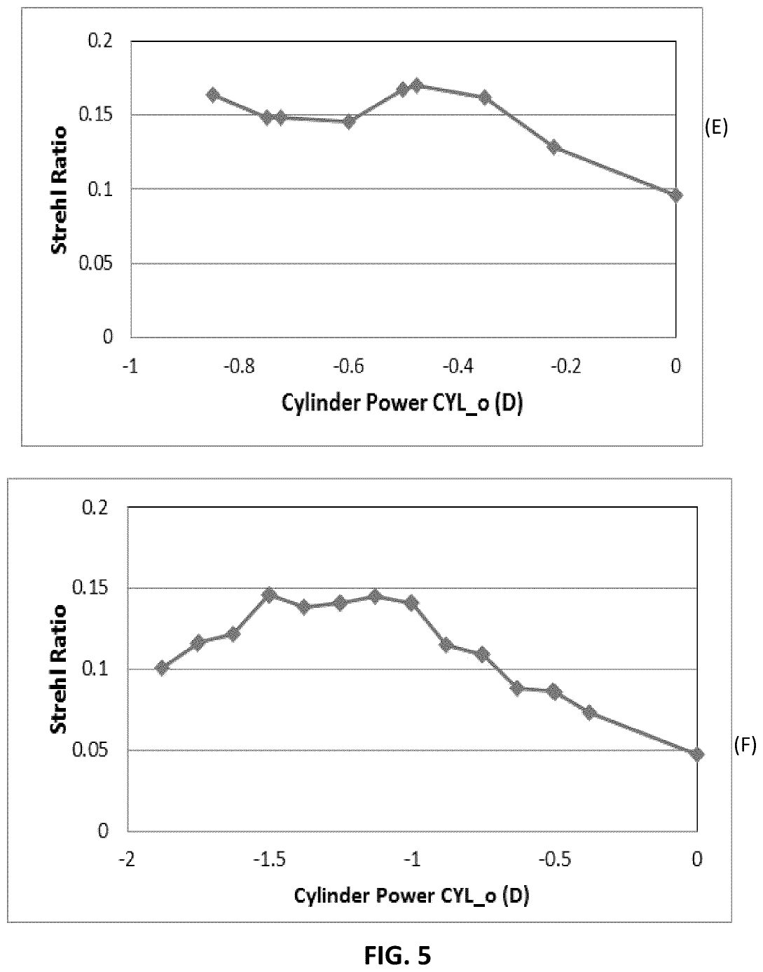

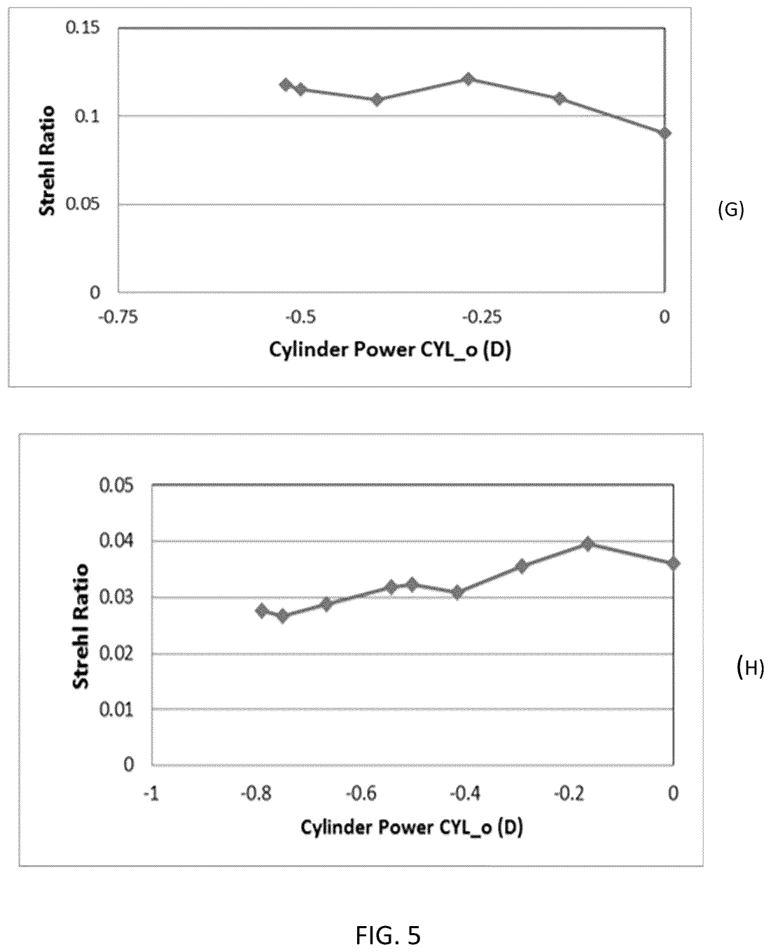

FIG. 4 shows calculated Strehl ratio of normalized point-spread function of 4 individual eyes as a function of cylinder powers. Clearly, there is a unique and unambiguous cylinder power that offers the best vision according to objective optimization. Under this circumstance, the confidence for the objectively optimized cylinder power CYL_o and cylinder angle AXIS_o should be high and without any doubt. Thus, the confidence level is considered high if 1) the profile has one significant peak located around the objective cylinder power (CYL_o) for the best optical quality, and 2) eye's Strehl ratio is significantly reduced with reduced cylinder power CYL_o. Strehl ratio for each eye was calculated from its residual aberrations. The confidence level is considered low in other situations as shown in FIG. 5 for the 4 other eyes. It is shown that there is a range of cylinder powers that offer similar Strehl Ratio, and Cylinder power with the highest Strehl ratio may not be the best cylinder correction. In one embodiment, a range of cylinder power with the objective cylinder power (CYL_o) for at least some eyes is provided. Additional optimization of cylinder power with feedback from patient would be necessary. Additionally, if the Strehl Ratio of the eye is low as shown in FIG. 4-H, Strehl ratio may have problems representing objective quality of corrected eye.

In another embodiment, the quality metrics for measuring the confidence level is the Strehl ratio of a calculated point spread function of the eye from residual aberrations under the optimized objective sphero-cylindrical correction. The confidence level is considered high if the Strehl ratio is larger than a specified threshold value, and low if the Strehl ratio is below the specified threshold value. In one embodiment, the specified threshold value for Strehl ratio is 0.20. In another embodiment, the specified threshold value for Strehl ratio depends on pupil size of the tested eye.

In yet another embodiment, the quality metrics for measuring the confidence level is displayed as a plurality of calculated retinal images of an acuity chart for a plurality of objective cylinder power (CYL_o). Each of the calculated retinal images represents the best optimized vision for each objective cylinder powers selected around the objective cylinder power (CYL_o). The confidence level and best optimized objective cylinder power (CYL_o) can be further determined by a human operator in reviewing the displayed retinal images of an acuity chart.

In one embodiment, the mode for the subjective determination of spherical power only is elected for the subjective refraction 35 in FIG. 3 if the confidence level is high, and the subjective refraction involves in determining a subjective spherical power SPH_s subjectively only and generating a refractive prescription for the eye that includes the subjective spherical power SPH_s, the objective cylinder power CYL_o, the objective cylinder axis AXIS_o.

In another embodiment, the mode for subjective determination of sphere power and cylinder power is elected for the subjective refraction 36 in FIG. 3 if the confidence level is low, and the objective cylinder power is either subjectively validated or updated with a new CYL_s in the subjective refraction, which involves in subjective optimization of the cylinder power with patient's subjective feedback.

In one embodiment, the objective aberrometer module comprises a principle or device chosen from the group consisting of: a Hartmann-Shack sensor, a laser ray tracing device, a spatially resolved refractometer, Talbot-Moire interferometry, skiascopic phase difference, and Tscherning principle.

In another embodiment, the objective refraction device include an autorefractor that is capable of generating a quality metrics for measuring the confidence level in the objectively determined cylinder power and cylinder axis, and the autorefractor can perform and generate a profile of quality of vision as a function of a plurality of cylinder powers near the objective cylinder power (CYL_o).

There are at least three advantages with the improved approach for optimizing refractive correction of human eyes in FIG. 3 according to the present invention through introducing a quality metrics for measuring the confidence level in the objectively determined cylinder power and cylinder axis and using the quality metrics to guide a subjective refraction with a phoropter in a plurality of modes.

First, it identifies majority of eyes (80% or more in the population) that can take advantages of the new approach in FIG. 1 if the confidence level in the objectively determined cylinder power CYL_o and cylinder axis (AXIS_o) is high. Subjective refraction of these eyes only involving in subjective determination of a spherical power SPH_s. This makes the refraction process significantly less time-consuming, independent of skills of the administrator, and allows for high-resolution/high-definition prescriptions for vast majority of eyes.

Second, it identify some eyes (20% or less) that may need to using the conventional manifest refraction that involving subjective optimization of both spherical power and cylinder power if the high confidence level in the objectively determined cylinder power CYL_o and cylinder axis (AXIS_o) is low. This will prevent erroneous prescriptions for these eyes if the approach in FIG. 2 is used, and can eliminate pains of patients for wearing the wrong eyeglasses, and offer savings by reducing eyeglasses rework.

Third, the quality metrics for measuring the confidence level in the objectively determined cylinder power and cylinder axis as shown in FIG. 4 provides new means to improve the traditional manifest rarefaction by 1) narrowing the search range for determining cylinder power subjectively, 2) providing a warning that a range of cylinder power can lead to similar vision outcomes. Operators of the subjective refraction process can then design the appropriate strategies to find the best cylinder power subjectively for the final refractive prescription.

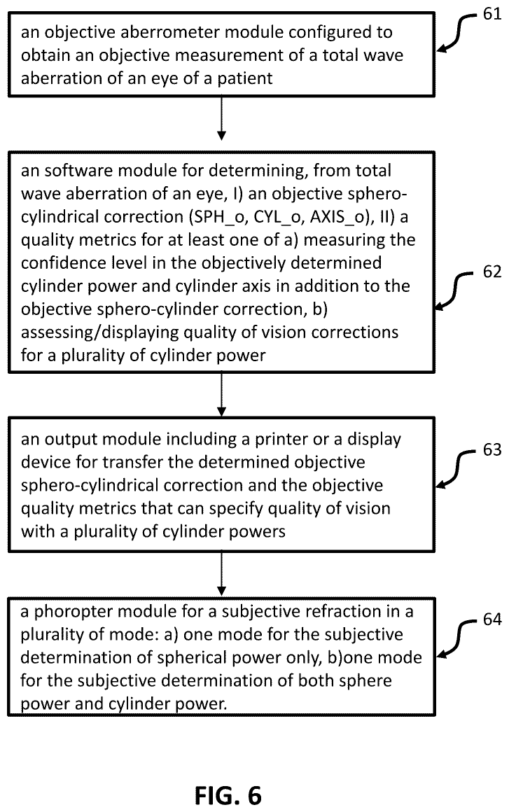

In some embodiments, FIG. 6 shows a system for determining refractive corrections of human eyes that include a wavefront device and a phoropter according to the present invention. The system comprises 1) an objective aberrometer module 61 configured to obtain an objective measurement of a total wave aberration of an eye of a patient, and the objective measurement does not involve responses from the patient, 2) an software module 62 for determining 2a) an objective sphero-cylindrical correction that includes an objective spherical power (SPH_o), an objective cylinder power (CYL_o), an objective cylinder axis (AXIS_o), 2b) a quality metrics for at least one of I) measuring the confidence level in the objectively determined cylinder power and cylinder axis in addition to the objective sphero-cylinder correction, II) assessing/displaying quality of vision corrections for a plurality of cylinder power.

In one embodiments, the quality metrics for measuring the confidence level in the determined objective cylinder power and cylinder axis is measured by one of the followings: I) a profile of Strehl ratio as a function of a plurality of cylinder powers near the objective cylinder power (CYL_o), II) a Strehl ratio of a calculated point spread function of the eye from residual aberrations under the optimized objective sphero-cylindrical correction, III) a plurality of calculated retinal images of a acuity chart for a plurality of cylinder power CYL_o and each of the calculated retinal image represents the best optimized vision for each objective cylinder power around the objective cylinder power (CYL_o).

In another embodiment, the system in FIG. 6 further include an output module 63 such as a printer or a display device for transfer the determined objective sphero-cylindrical correction as well as the quality metrics in addition to the objective sphero-cylinder correction.

In yet another embodiment, the system in FIG. 6 further includes a phoropter module 64 for a subjective refraction in a plurality of modes: a) one mode for the subjective determination of spherical power only, b) another mode for subjective determination of both sphere power and cylinder power.

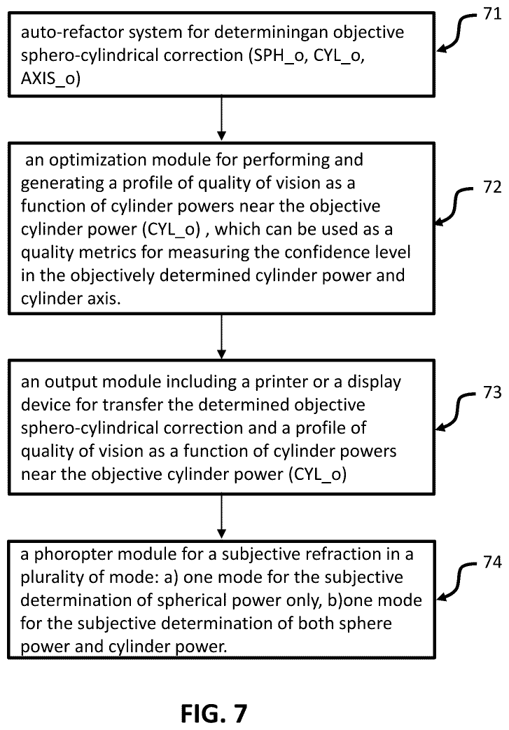

In some embodiments, FIG. 7 shows another system for determining refractive corrections of human eyes that include a improved auto-refractor and a phoropter according to the present invention. The improved auto-refactor system for determining refractive correction of human eyes comprise: 1) a measurement module 71 configured to obtain an objective measurement of an objective sphero-cylindrical correction that includes an objective spherical power (SPH_o), an objective cylinder power (CYL_o), an objective cylinder axis (AXIS_o), 2) an optimization module 72 for performing and generating a profile of quality of vision as a function of a plurality of cylinder powers near the objective cylinder power (CYL_o).

In one embodiment, the system in FIG. 7 further include an output module 73 such as a printer or a display device for transfer the determined objective sphero-cylindrical correction as well as the generated a profile of quality of vision as a function of a plurality of cylinder powers near the objective cylinder power.

In another embodiment, the system in FIG. 7 further include a phoropter module 74 for a subjective refraction in a plurality of mode: a) one mode for the subjective determination of spherical power only, b) one mode for subjective determination of both sphere power and cylinder power.

Reference has been made in detail to embodiments of the disclosed invention, one or more examples of which have been illustrated in the accompanying figures. Each example has been provided by way of explanation of the present technology, not as a limitation of the present technology. In fact, while the specification has been described in detail with respect to specific embodiments of the invention, it will be appreciated that those skilled in the art, upon attaining an understanding of the foregoing, may readily conceive of alterations to, variations of, and equivalents to these embodiments. For instance, features illustrated or described as part of one embodiment may be used with another embodiment to yield a still further embodiment. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only and is not intended to limit the invention.

What is claimed is:

1. A method for determining refractive corrections of human eyes, the method comprising the steps of:
obtaining an objective refraction of an eye of a patient using an objective refraction device, wherein the objective refraction does not involve any subjective feedback from tested subjects and it includes at least an objective sphero-cylinder prescription consisting of an objective spherical power (SPH_o), an objective cylinder power (CYL_o), and an objective cylinder axis (AXIS_o);
providing a range of cylinder power with the objective cylinder power (CYL_o) for at least some eyes or determining a quality metrics for at least one of 1) measuring the confidence level in the objectively determined cylinder power and cylinder axis in addition to the objective sphero-cylinder correction, 2) assessing/displaying quality of vision corrections for a plurality of cylinder power;
using the quality metrics or the provided a range of cylinder power with the objective cylinder power (CYL_o) for at least some eyes to perform a subjective refraction with a phoropter in a plurality of modes: I) one mode for the subjective determination of spherical power only, II) one mode for the subjective determination of both sphere power and cylinder power.

2. The method of claim 1 wherein the objective refraction device is a wavefront aberrometer that provides an objective measurement of a total wave aberration of an eye of a patient, wherein the total wave aberration includes the objective sphero-cylindrical correction as well as eye's residual aberrations that are not corrected by the objective sphero-cylindrical correction.

3. The method of claim 2 wherein the quality metrics is measured by a profile of Strehl ratio of eye's point-spread function as a function for a plurality of cylinder powers near the objective cylinder power (CYL_o) from which a range of cylinder power with the objective cylinder power (CYL_o) is determined for at least some eyes, wherein Strehl ratio is the peak intensity of normalized point-spread function and calculated from the residual aberration.

4. The method of claim 3 wherein the quality metrics is further displayed for an operator to view and determine the confidence level in the objectively determined cylinder power and cylinder axis.

5. The method of claim 3 wherein the confidence level is high if the profile has one significant peak located around the objective cylinder power (CYL_o) plus having Strehl Ratio significantly reduced with decreased objective cylinder power, and the confidence level is low in other situations.

6. The method of claim 5 wherein the confidence level is determined either automatically with an algorithm or by an operator subjectively.

7. The method of claim 2 wherein the quality metrics is measured by a Strehl ratio of a calculated point spread function of the eye from residual aberrations under the optimized objective sphero-cylindrical correction.

8. The method of claim 7 wherein the confidence level is considered high if the Strehl ratio is larger than a specified threshold value, and low if the Strehl ratio is below the specified threshold value.

9. The method of claim 8 wherein the specified threshold value for Strehl ratio is 0.20.

10. The method of claim 8 wherein the specified threshold value for Strehl ratio depends on pupil size of the tested eye.

11. The method of claim 2 where the quality metrics is displayed as a plurality of calculated retinal images of a acuity chart for a plurality of cylinder powers, wherein each of the calculated retinal image represents the best optimized vision for each objective cylinder powers around the objective cylinder power (CYL_o).

12. The method of claim 11 wherein the confidence level and the best optimized objective cylinder power is determined by a human operator in reviewing the displayed retinal images of an acuity chart.

13. The method of claim 1 elects the mode for the subjective determination of spherical power only if the confidence level is high, and the subjective refraction involves in subjectively determining a subjective spherical power SPH_s and generating a refractive prescription for the eye including the subjective spherical power SPH_s, the objective cylinder power CYL_o, the objective cylinder axis AXIS_o.

14. The method of claim 1 elects the mode for the subjective determination of both sphere power and cylinder power if the confidence level is low or a range of cylinder power with the objective cylinder power (CYL_o) for at least some eyes, and the objective cylinder power is either subjectively validated or updated with a new CYL_s in the subjective refraction that involves in subjective optimization of the cylinder power with patient's subjective feedback.

15. The method of claim 2 wherein the objective aberrometer module comprises a principle or device chosen from the group consisting of: a Hartmann-Shack sensor, a laser ray tracing device, a spatially resolved refractometer, Talbot-Moire interferometry, skiascopic phase difference, and Tscherning principle.

16. The method of claim 1 wherein the objective refraction device include an autorefractor that is capable of generating a quality metrics for measuring the confidence level in the objectively determined cylinder power and cylinder axis, wherein the autorefractor generates a profile of quality of vision as a function of a plurality of cylinder powers near the objective cylinder power (CYL_o).

17. A system for determining refractive corrections of human eyes, comprising:
an objective aberrometer module configured to obtain an objective measurement of a total wave aberration of an eye of a patient, wherein the objective measurement does not involve responses from the patient;
an software module for determining from the total wave aberration of an eye, I) an objective sphero-cylindrical correction that includes an objective spherical power (SPH_o), an objective cylinder power (CYL_o), an objective cylinder axis (AXIS_o), II) a range of cylinder power with the objective cylinder power (CYL_o) for at least some eyes as a part of objective refraction or a quality metrics for at least one of a) measuring the confidence level in the objectively determined cylinder power and cylinder axis in addition to the objective sphero-cylinder correction, b) assessing/displaying quality of vision corrections for a plurality of cylinder power.

18. The system of claim 17 wherein the quality metrics is measured by at least one of the followings: I) a profile of Strehl ratio as a function of cylinder powers near the objective cylinder power (CYL_o), II) a plurality of calculated retinal images of a acuity chart for a plurality of cylinder powers, wherein each of the calculated retinal image represents the best optimized vision for different objective cylinder powers around the objective cylinder power (CYL_o).

19. The system of claim 17 further include an output module including a printer or a display device for transfer the determined objective sphero-cylindrical correction as well as the quality metrics.

20. The system of claim 17 further include a phoropter module for a subjective refraction in a plurality of mode: a) one mode for the subjective determination of spherical power only, b) one mode for the subjective determination of both sphere power and cylinder power.

21. The system of claim 20 elects the mode for the subjective determination of spherical power only if the confidence level is high, and the subjective refraction involves in subjectively determining a subjective spherical power SPH_s and generating a prescription for the eye including the subjective spherical power SPH_s, the objective cylinder power CYL_o, the objective cylinder axis AXIS_o.

22. The system of claim 20 elects the mode for the subjective determination of both sphere power and cylinder power if the confidence level is low or a range of cylinder power with the objective cylinder power (CYL_o) for at least some eyes, and the objective cylinder power is either subjectively validated or updated with a new CYL_s in the subjective refraction involving in subjective optimization of the cylinder power with patient's subjective feedback.

23. An improved auto-refactor system for determining refractive correction of human eyes, comprising:
a measurement module configured to obtain an objective measurement of an objective sphero-cylindrical correction that includes an objective spherical power (SPH_o), an objective cylinder power (CYL_o), an objective cylinder axis (AXIS_o);
an optimization module for providing a range of cylinder power with the objective cylinder power (CYL_o) for at least some eyes or performing and generating a profile of quality of vision as a function of a plurality of cylinder powers near the objective cylinder power (CYL_o).

24. The system of claim 23 further include an output module including a printer or a display device for transfer the determined objective sphero-cylindrical correction as well as the generated a profile of quality of vision as a function of a plurality of cylinder powers near the objective cylinder power.

25. The system of claim 23 further include a phoropter module for a subjective refraction in a plurality of mode: a) one mode for the subjective determination of spherical power only, b) one mode for the subjective determination of both sphere power and cylinder power.

26. The system of claim 25 elects the mode for the subjective determination of spherical power only if the confidence level is high, and the subjective refraction involves in subjectively determining a subjective spherical power SPH_s and generating a prescription for the eye including the subjective spherical power SPH_s, the objective cylinder power CYL_o, the objective cylinder axis AXIS_o.

27. The system of claim 25 elects the mode for the subjective determination of both sphere power and cylinder power if the confidence level is low or a range of cylinder power with the objective cylinder power (CYL_o) for at least some eyes, and the objective cylinder power is either subjectively validated or updated with a new CYL_s in the subjective refraction involving in subjective optimization of the cylinder power with patient's subjective feedback.
